(19) United States
(12) Patent Application Publication
(10) Pub. No.: US 2010/0269212 A1
(43) Pub. Date: Oct. 21, 2010

(54) GENETIC LOCI ASSOCIATED WITH FUSARIUM EAR ROT (FKR) RESISTANCE IN MAIZE AND GENERATION OF IMPROVED FKR RESISTANT MAIZE INBRED LINES

(75) Inventors: Nathan J. VanOpdorp, Geneseo, IL (US); Klaus L. Koehler, West Lafayette, IN (US)

Correspondence Address: DOW AGROSCIENCES LLC, 9330 ZIONSVILLE RD, INDIANAPOLIS, IN, 46268, US

(73) Assignee: Agrigenetics, Inc.

(21) Appl. No.: 12/755,619

(22) Filed: Apr. 7, 2010

Related U.S. Application Data
(60) Provisional application No. 61/170,870, filed on Apr. 20, 2009.

(51) Int. Cl. A01H 1/02; A01H 1/04; C12Q 1/68

(52) U.S. Cl. 800/265; 435/6

(57) ABSTRACT

Methods of genetic marker assisted selection of Fusarium Ear Rot resistance in maize plants include isolating DNA from the maize plant. The DNA is then assessed to identify plants having one or more of the SSR genetic markers selected from the group consisting of phi333597, umc2013, umc1350, dup013, umc1665, and umc1412. Plants having the Fusarium Ear Rot resistance are then selected. Methods of identifying a first maize plant or germplasm that displays improved resistance to FKR include detecting in the first maize plant or germplasm at least one allele of one or more genetic markers associated with the FKR resistance selected from the group consisting of phi333597, umc2013, umc1350, dup013, umc1665, and umc1412.

This application claims the benefit of U.S. Provisional Application 61/170,870 filed on Apr. 20, 2009.

### FIELD OF THE INVENTION

The invention relates to methods for identifying maize plants that are resistant to fusarium ear rot (FKR) and to methods using molecular genetic markers to identify, select and/or construct FKR resistant maize plants.

### BACKGROUND OF THE INVENTION

Fusarium verticillioides is a pathogen of maize establishing long-term associations with the plant (Baba-Moussa, 1998; Pitt and Hocking, 1999) which can infect maize at all stages of plant development, causing grain rot and fumonisin accumulation during pre-harvest and post harvest periods (Munkvold and Desjardins, 1997). Symptomless infection can exist throughout the plant in leaves, stems, roots, and kernels. The presence of the fungus is in many cases ignored because it does not cause visible damage to the plant (Munkvold and Desjardins, 1997) During this symptomless phase, most consider the endophytic hyphae to be latent, quiescent, or dormant and suggested that symptomless infected plants were non-hosts that served the purpose of over wintering the fungus, from which it produced conidia during its saprophytic stage. (Bacon et al, 2001) F. verticillioides is labeled as a microbial endophyte because it actively colonizes and establishes a long term association with the host and even a lifelong symptomless association can be established.

F. verticillioides can be transmitted vertically and horizontally to the next generation of plants via clonal infection of seeds and plant debris. (Bacon et al, 2001) In vertical transmission the pathogen will go from the infected seed that was planted, through the plant, and infect the seed on the ear produced by the plant. In horizontal transmission the airborne and rain splashed conidia produced by the plant debris in the field, will land on the silk and eventually contact the ear. (Fandohan et al, 2003) It may also be introduced to the stem and cob of the plant via insects. (Munkvold and Carlton, 1997) Once F. verticillioides is present, it is most easily identified by the presence of ear rot. Although ear rot is a good indicator that F. verticillioides is present, it is very common for the pathogen to be present with no visual damage seen on the kernels or maize ears.

Many factors can influence the severity of F. verticillioides and in turn the severity of fumonisin levels such as climate, temperature, and cultivation practices. Studies have shown that the difference in rainfall levels preceding the month before harvest effect the levels of fumonisin and the presence of Fusarium. (Ono et al 1999) In this study the heavier rainfalls (202 mm) resulted in higher levels of fumonisin as compared to lower levels of rainfall (92 mm). It has also been shown that dry weather at or just prior to pollination of maize might be an important factor for fumonisin production in maize (Shelby et al, 1994). Temperature can also play a role in the growth rate of F. verticillioides, as research has shown that growth rates of F. verticillioides was higher at a temperature of 25° C. when compared to lower growth at 15° C. (Velluti et al, 2000). It was also found that at constant temperature, water activity can play an important role in the infection and fumonisin accumulation of maize. With the different factors influencing Fusarium infection, it can be very difficult to phenotype and screen for the disease correctly.

The presence of F. verticillioides in maize is most easily identified by the presence of rot or mold on the maize ear and kernels referred to as Fusarium Ear Rot (FKR). The ear rot is characterized by cottony mycelium growth that typically occurs on a few kernels or is limited to certain parts of the ear. Mycelium is generally white, pale pink or pale lavender. Infected kernels typically display white streaking (also known as ‘starburst’ symptoms) on the pericarp and often germinate on the cob. Typically, infection occurs close to ear tips and is commonly associated with damage and injury caused by ear borers. Under severe infestation, the entire ear appears withered and is characterized by mycelium growth between kernels. (CIMMYT, Maize Doctor) This ear rot and mold can result in the loss of money for seed producers and grain producers as it will result in lower quality grain, but more concerning is the ear rot indicates that toxins called fumonisins are possibly accumulating in the grain.

Fumonisin production in the maize kernels is definitely not as easy to detect as ear rot symptoms, but the mycotoxin is definitely more concerning because it can be harmful to horses, pigs, and even humans. Fumonisin can be produced by several species of Fusarium but the two species that are the most prolific fumonisin producers are F. verticillioides and F. proliferatum, and maize is the product in which fumonisins are most abundant (Shephard et al., 1996). As of 2002, a total of 28 fumonisin analogs have been identified and characterized (Rheeder et al., 2002) with FB1, FB2, and FB3 being the most abundantly found in maize foods and feeds. Although ear rot is not a precise way to determine the fumonisin level present in the grain, it is a good visual indicator that the plant has been infected with Fusarium, and fumonisin accumulation in the ear is highly probable.

Control, prevention, and detection of the endophytic infections of F. verticillioides in corn is difficult, due to the intercellular nature of F. verticillioides. Chemical controls are highly unlikely, as the applications of systemic fungicides are impossible during later stages of plant growth. The fungus is a systemic seed-borne infection, so conventional fungicides used as seed treatments are also ineffective. (Bacon, et al., 2001) Breeding efforts are able to produce cultivars that have been selected for enhanced resistance to FKR. Through the use of a disease screening nurseries, new cultivars can be selected for increased resistance levels. Recent studies have detected multiple genes in maize that are correlated with the resistance to F. verticillioides and reduction in Fumonisin levels. (Robertson, et al., 2006) These resistance genes or QTLs could then be used in conjunction with normal plant breeding selections, which is a technique known as Marker Assisted Breeding (MAS), and this would help enhance the quality of resistance selected for by capturing the QTLs of interest in each new maize line.

The use of an endophytic bacterium such as Bacillus mojavensis or Bacillus subtillis, has also shown promise in the control of Fusarium species. B. subtillis (Ehrenberg) Cohn, is an isolate of an endophytic bacterium that shows great promise in the control and reduction of mycotoxin accumulation during the growth of maize plants endophytically infected with F. verticillioides (Bacon et al., 2001) Biological controls like B. subtillis can play a role in the biotechnology market and/or industrial applications. Other attempts to control F. verticillioides and reduce fumonisin levels include the use of Plantpro45™ as a biocompatible control of the fungus (Yates et al., 2000) and the use of non-producing strains of F. verticillioides aiming to minimize fumonisin levels in maize (Plattner et al., 2000) The control of insects such as European core bore, armyworms, and earworms through the use of maize tissue expressing proteins such as Cry1F and Cry1A(b), or through insecticide applications, can reduce the effects of Fusarium as well. As a rule, control of F. verticillioides in maize and reduction in accumulation of fumonisin is very difficult, yet highly important to the quality of future maize cultivars.

### BRIEF SUMMARY OF THE INVENTION

The following embodiments are described in conjunction with systems, tools and methods which are meant to be exemplary and illustrative, and not limiting in scope.

According to a particular embodiment of the invention, a method of genetic marker assisted selection of Fusarium Ear Rot resistance in maize plants includes isolating DNA from the maize plant. The DNA is then assessed to identify plants having one or more of the SSR genetic markers selected from the group consisting of phi333597, umc2013, umc1350, dup013, umc1665, and umc1412. Plants having the Fusarium Ear Rot resistance are then selected.

In another embodiment, the DNA is further assessed to identify plants having a marker within 1 centimorgan of one or more of the SSR genetic markers selected from the group consisting of phi333597, umc2013, umc1350, dup013, umc1665, and umc1412.

Yet another embodiment of the invention includes a method of identifying a first maize plant or germplasm that displays improved resistance to FKR. The method includes detecting in the first maize plant or germplasm at least one allele of one or more genetic markers associated with the FKR resistance selected from the group consisting of phi333597, umc2013, umc1350, dup013, umc1665, and umc1412.

In addition to the exemplary aspects and embodiments described above, further aspects and embodiments will become apparent in view of the following descriptions.

### BRIEF DESCRIPTION OF THE DRAWINGS

FIG. 1 shows graphs of ear ratings for population NV14/NV14FR F2;

FIG. 2 shows graphs of ear ratings for population NV35/NV14FR F2;

FIG. 3 shows analysis of marker phi333597;

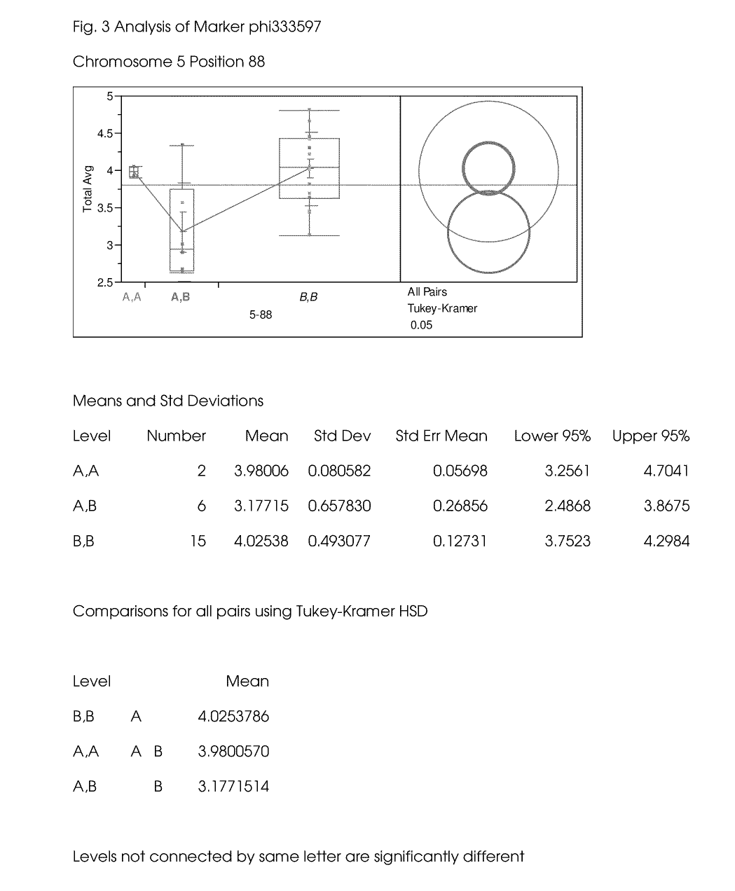

FIG. 4 shows analysis of marker umc1485;

FIG. 5 shows analysis of marker umc2013;

FIG. 6 shows analysis of marker umc1350;

FIG. 7 shows analysis of marker dup013;

FIG. 8 shows analysis of marker umc1665; and

FIG. 9 shows analysis of marker umc1412.

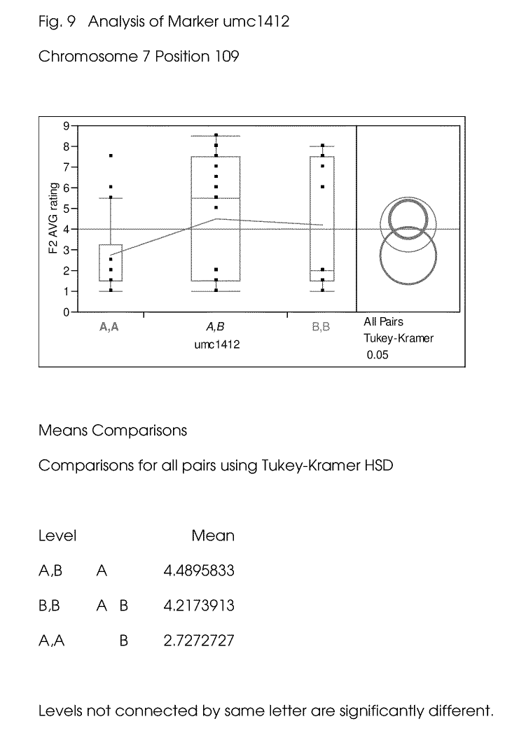

### DETAILED DESCRIPTION OF THE INVENTION

In the description and tables which follow, a number of terms are used. In order to provide a clear and consistent understanding of the specification and claims, including the scope to be given such terms, the following definitions are provided:

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant. Thus, the term “maize plant” includes whole maize plants, maize plant cells, maize plant protoplast, maize plant cell or maize tissue culture from which maize plants can be regenerated, maize plant calli, maize plant clumps and maize plant cells that are intact in maize plants or parts of maize plants, such as maize seeds, maize pods, maize flowers, maize cotyledons, maize leaves, maize stems, maize buds, maize roots, maize root tips and the like.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, that can be cultured into a whole plant.

The term “allele” refers to one of two or more different nucleotide sequences that occur at a specific locus. For example, a first allele can occur on one chromosome, while a second allele occurs on a second homologous chromosome, e.g., as occurs for different chromosomes of a heterozygous individual, or between different homozygous or heterozygous individuals in a population. A “favorable allele” is the allele at a particular locus that confers, or contributes to, an agronomically desirable phenotype, e.g., resistance to FKR, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying disease-prone plants. A favorable allelic form of a chromosome segment is a chromosome segment that includes a nucleotide sequence that contributes to superior agronomic performance at one or more genetic loci physically located on the chromosome segment. “Allele frequency” refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An allele “positively” correlates with a trait when it is linked to it and when presence of the allele is an indictor that the desired trait or trait form will occur in a plant comprising the allele. An allele negatively correlates with a trait when it is linked to it and when presence of the allele is an indicator that a desired trait or trait form will not occur in a plant comprising the allele.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal region where a polymorphic nucleic acid, trait determinant, gene or marker is located. Thus, for example, a “gene locus” is a specific chromosome location in the genome of a species where a specific gene can be found. The term “quantitative trait locus” or “QTL” refers to a polymorphic genetic locus with at least two alleles that differentially affect the expression of a phenotypic trait in at least one genetic background, e.g., in at least one breeding population or progeny.

The terms “marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. In some aspects, the present invention provides marker loci correlating with resistance to FKR in maize. Each of the identified markers is expected to be in close physical and genetic proximity (resulting in physical and/or genetic linkage) to a genetic element, e.g., a QTL, that contributes to resistance.

“Genetic markers” are nucleic acids that are polymorphic in a population and where the alleles of which can be detected and distinguished by one or more analytic methods, e.g., RFLP, AFLP, isozyme, SNP, SSR, and the like. The terms “genetic marker” and “molecular marker” refer to a genetic locus (a “marker locus”) that can be used as a point of reference when identifying a genetically linked locus such as a QTL. Such a marker is also referred to as a QTL marker. The term also refers to nucleic acid sequences complementary to the genomic sequences, such as nucleic acids used as probes.

Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

As used herein, the term “maize” means Zea mays or corn and includes all plant varieties that can be bred with corn, including wild maize species. More specifically, corn plants from the species Zea mays and the subspecies Zea mays L. ssp. Mays can be genotyped using the compositions and methods of the present invention. In an additional aspect, the corn plant is from the group Zea mays L. subsp. mays Indentata, otherwise known as dent corn. In another aspect, the corn plant is from the group Zea mays L. subsp. mays Indurata, otherwise known as flint corn. In another aspect, the corn plant is from the group Zea mays L. subsp. mays Saccharata, otherwise known as sweet corn. In another aspect, the corn plant is from the group Zea mays L. subsp. mays Amylacea, otherwise known as flour corn. In a further aspect, the corn plant is from the group Zea mays L. subsp. mays Everta, otherwise known as pop corn. Zea or corn plants that can be genotyped with the compositions and methods described herein include hybrids, inbreds, partial inbreds, or members of defined or undefined populations.

A “genetic map” is a description of genetic linkage relationships among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a physical map of the genome refers to absolute distances (for example, measured in base pairs or isolated and overlapping contiguous genetic fragments, e.g., contigs). A physical map of the genome does not take into account the genetic behavior (e.g., recombination frequencies) between different points on the physical map.

A “genetic recombination frequency” is the frequency of a crossing over event (recombination) between two genetic loci. Recombination frequency can be observed by following the segregation of markers and/or traits following meiosis. A genetic recombination frequency can be expressed in centimorgans (cM), where one cM is the distance between two genetic markers that show a 1% recombination frequency (i.e., a crossing-over event occurs between those two markers once in every 100 cell divisions).

As used herein, the term “linkage” is used to describe the degree with which one marker locus is “associated with” another marker locus or some other locus (for example, a resistance locus).

As used herein, the linkage relationship between a molecular marker and a phenotype is given as a “probability” or “adjusted probability.” The probability value is the statistical likelihood that the particular combination of a phenotype and the presence or absence of a particular marker allele is random. Thus, the lower the probability score, the greater the likelihood that a phenotype and a particular marker will co-segregate. In some aspects, the probability score is considered “significant” or “nonsignificant.” In some embodiments, a probability score of 0.05 (p=0.05, or a 5% probability) of random assortment is considered a significant indication of co-segregation. However, the present invention is not limited to this particular standard, and an acceptable probability can be any probability of less than 50% (p=0.5). For example, a significant probability can be less than 0.25, less than 0.20, less than 0.15, or less than 0.1.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Linked loci co-segregate more than 50% of the time, e.g., from about 51% to about 100% of the time. The term “physically linked” is sometimes used to indicate that two loci, e.g., two marker loci, are physically present on the same chromosome.

Advantageously, the two linked loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time.

The phrase “closely linked,” in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful in the present invention when they demonstrate a significant probability of co-segregation (linkage) with a desired trait (e.g., pathogenic resistance). For example, in some aspects, these markers can be termed linked QTL markers. In other aspects, especially useful molecular markers are those markers that are linked or closely linked to QTL markers.

In some aspects, linkage can be expressed as any desired limit or range. For example, in some embodiments, two linked loci are two loci that are separated by less than 50 cM map units. In other embodiments, linked loci are two loci that are separated by less than 40 cM. In other embodiments, two linked loci are two loci that are separated by less than 30 cM. In other embodiments, two linked loci are two loci that are separated by less than 25 cM. In other embodiments, two linked loci are two loci that are separated by less than 20 cM. In other embodiments, two linked loci are two loci that are separated by less than 15 cM. In some aspects, it is advantageous to define a bracketed range of linkage, for example, between 10 and 20 cM, or between 10 and 30 cM, or between 10 and 40 cM.

The more closely a marker is linked to a second locus, the better an indicator for the second locus that marker becomes. Thus, in one embodiment, closely linked loci such as a marker locus and a second locus (e.g., a QTL marker) display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a QTL marker) display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

In some aspects, for example in the context of the present invention, generally the genetic elements located within a single chromosome interval are also genetically linked, typically within a genetic recombination distance of, for example, less than or equal to 20 centimorgan (cM), or alternatively, less than or equal to 10 cM. That is, two genetic elements within a single chromosome interval undergo recombination at a frequency of less than or equal to 20% or 10%. In one aspect, any marker of the invention is linked (genetically and physically) to any other marker that is at or less than 50 cM distant. In another aspect, any marker of the invention is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

The term “crossed” or “cross” in the context of this invention means the fusion of gametes via pollination to produce progeny (e.g., cells, seeds or plants). The term encompasses both sexual crosses (the pollination of one plant by another) and selfing (self-pollination, e.g., when the pollen and ovule are from the same plant).

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor. Traditionally, a “subline” has been derived by inbreeding the seed from an individual maize plant selected at the F3 to F5 generation until the residual segregating loci are “fixed” or homozygous across most or all loci. Commercial maize varieties (or lines) are typically produced by aggregating (“bulking”) the self-pollinated progeny of a single F3 to F5 plant from a controlled cross between 2 genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. In the context of the invention, marker-based sublines, that differ from each other based on qualitative polymorphism at the DNA level at one or more specific marker loci, are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (yield, resistance, etc.).

An “elite line” or “elite strain” is an agronomically superior line that has resulted from many cycles of breeding and selection for superior agronomic performance. Numerous elite lines are available and known to those of skill in the art of maize breeding. An “elite population” is an assortment of elite individuals or lines that can be used to represent the state of the art in terms of agronomically superior genotypes of a given crop species, such as maize Similarly, an “elite germplasm” or elite strain of germplasm is an agronomically superior germplasm, typically derived from and/or capable of giving rise to a plant with superior agronomic performance, such as an existing or newly developed elite line of maize.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

The term “transgenic plant” refers to a plant that comprises within its cells a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been altered by the presence of heterologous nucleic acid including those transgenic organisms or cells initially so altered, as well as those created by crosses or asexual propagation from the initial transgenic organism or cell. The term “transgenic” as used herein does not encompass the alteration of the genome (chromosomal or extra-chromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

The term “genetic element” or “gene” refers to a heritable sequence of DNA, i.e., a genomic sequence, with functional significance. The term “gene” can also be used to refer to, e.g., a cDNA and/or a mRNA encoded by a genomic sequence, as well as to that genomic sequence.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. A “haplotype” is the genotype of an individual at a plurality of genetic loci. Typically, the genetic loci described by a haplotype are physically and genetically linked, i.e., on the same chromosome segment.

The terms “phenotype,” or “phenotypic trait” or “trait” refers to one or more trait of an organism. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait.” In other cases, a phenotype is the result of several genes. A “quantitative trait loci” (QTL) is a genetic domain that is polymorphic and effects a phenotype that can be described in quantitative terms, e.g., height, weight, oil content, days to germination, disease resistance, etc, and, therefore, can be assigned a “phenotypic value” which corresponds to a quantitative value for the phenotypic trait. A QTL can act through a single gene mechanism or by a polygenic mechanism. A “molecular phenotype” is a phenotype detectable at the level of a population of (one or more) molecules. Such molecules can be nucleic acids such as genomic DNA or RNA, proteins, or metabolites. For example, a molecular phenotype can be an expression profile for one or more gene products, e.g., at a specific stage of plant development, in response to an environmental condition or stress, etc. Expression profiles are typically evaluated at the level of RNA or protein, e.g., on a nucleic acid array or “chip” or using antibodies or other binding proteins.

The term “yield” refers to the productivity per unit area of a particular plant product of commercial value. For example, yield of maize is commonly measured in bushels of seed per acre or metric tons of seed per hectare per season. Yield is affected by both genetic and environmental factors. “Agronomics,” “agronomic traits,” and “agronomic performance” refer to the traits (and underlying genetic elements) of a given plant variety that contribute to yield over the course of growing season. Individual agronomic traits include emergence vigor, vegetative vigor, stress tolerance, disease resistance or tolerance, herbicide resistance, branching, flowering, seed set, seed size, seed density, standability, threshability and the like. Yield is, therefore, the final culmination of all agronomic traits.

A “set” of markers or probes refers to a collection or group of markers or probes, or the data derived therefrom, used for a common purpose, e.g., identifying maize plants with a desired trait (e.g., resistance to fusarium ear rot infection). Frequently, data corresponding to the markers or probes, or data derived from their use, is stored in an electronic medium. While each of the members of a set possess utility with respect to the specified purpose, individual markers selected from the set as well as subsets including some, but not all of the markers, are also effective in achieving the specified purpose.

The identification and selection of maize plants that show resistance to FKR using MAS can provide an effective and environmentally friendly approach to overcoming losses caused by this disease. The present invention provides maize marker loci that demonstrate statistically significant co-segregation with FKR resistance. Detection of these loci or additional linked loci can be used in marker assisted maize breeding programs to produce resistant plants, or plants with improved resistance to FKR. The linked SSR markers identified herein include phi333597, umc2013, umc1350, dup013, umc1665, and umc1412. Each of the SSR-type markers display a plurality of alleles that can be visualized as different sized PCR amplicons.

Methods for identifying maize plants or germplasm that carry preferred alleles of resistance marker loci are a feature of the invention. In these methods, any of a variety of marker detection protocols are used to identify marker loci, depending on the type of marker loci. Typical methods for marker detection include amplification and detection of the resulting amplified markers, e.g., by PCR, LCR, transcription based amplification methods, or the like. These include ASH, SSR detection, RFLP analysis and many others. Although particular marker alleles can show co-segregation with a disease resistance or susceptibility phenotype, it is important to note that the marker locus is not necessarily part of the QTL locus responsible for the resistance or susceptibility. For example, it is not a requirement that the marker polynucleotide sequence be part of a gene that imparts disease resistance (for example, be part of the gene open reading frame). The association between a specific marker allele with the resistance or susceptibility phenotype is due to the original “coupling” linkage phase between the marker allele and the QTL resistance or susceptibility allele in the ancestral maize line from which the resistance or susceptibility allele originated. Eventually, with repeated recombination, crossing over events between the marker and QTL locus can change this orientation. For this reason, the favorable marker allele may change depending on the linkage phase that exists within the resistant parent used to create segregating populations. This does not change the fact the genetic marker can be used to monitor segregation of the phenotype. It only changes which marker allele is considered favorable in a given segregating population.

Identification of maize plants or germplasm that include a marker locus or marker loci linked to a resistance trait or traits provides a basis for performing marker assisted selection of maize. Maize plants that comprise favorable markers or favorable alleles are selected for, while maize plants that comprise markers or alleles that are negatively correlated with resistance can be selected against. Desired markers and/or alleles can be introgressed into maize having a desired (e.g., elite or exotic) genetic background to produce an introgressed resistant maize plant or germplasm. In some aspects, it is contemplated that a plurality of resistance markers are sequentially or simultaneous selected and/or introgressed. The combinations of resistance markers that are selected for in a single plant is not limited, and can include any combination of identified markers, any markers linked to the identified markers, or any markers located within the QTL intervals defined herein.

As an alternative to standard breeding methods of introducing traits of interest into maize (e.g., introgression), transgenic approaches can also be used. In these methods, exogenous nucleic acids that encode traits linked to markers are introduced into target plants or germplasm. For example, a nucleic acid that codes for a resistance trait is cloned, e.g., via positional cloning and introduced into a target plant or germplasm.

Systems, including automated systems for selecting plants that comprise a marker of interest and/or for correlating presence of the marker with FKR resistance are also a feature of the invention. These systems can include probes relevant to marker locus detection, detectors for detecting labels on the probes, appropriate fluid handling elements and temperature controllers that mix probes and templates and/or amplify templates, and systems instructions that correlate label detection to the presence of a particular marker locus or allele.

A favorable allele of a marker is that allele of the marker that co-segregates with a desired phenotype (e.g., disease resistance). As used herein, a QTL marker has a minimum of one favorable allele, although it is possible that the marker might have two or more favorable alleles found in the population. Any favorable allele of that marker can be used advantageously for the identification and construction of FKR resistant maize lines. Optionally, one, two, three or more favorable allele(s) of different markers are identified in, or introgressed into a plant, and can be selected for or against during MAS. Desirably, plants or germplasm are identified that have at least one such favorable allele that positively correlates with resistance. Alternatively, a marker allele that co-segregates with disease susceptibility also finds use with the invention, since that allele can be used to identify and counter select disease-susceptible plants. Such an allele can be used for exclusionary purposes during breeding to identify alleles that negatively correlate with resistance, to eliminate susceptible plants or germplasm from subsequent rounds of breeding.

In some embodiments of the invention, a plurality of marker alleles are simultaneously selected for in a single plant or a population of plants. In these methods, plants are selected that contain favorable alleles from more than one resistance marker, or alternatively, favorable alleles from more than one resistance marker are introgressed into a desired maize germplasm. One of skill in the art recognizes that the simultaneous selection of favorable alleles from more than one disease resistance marker in the same plant is likely to result in an additive (or even synergistic) protective effect for the plant.

One of skill recognizes that the identification of favorable marker alleles is germplasm-specific. The determination of which marker alleles correlate with resistance (or susceptibility) is determined for the particular germplasm under study. One of skill recognizes that methods for identifying the favorable alleles are routine and well known in the art, and furthermore, that the identification and use of such favorable alleles is well within the scope of the invention. Furthermore still, identification of favorable marker alleles in maize populations other than the populations used or described herein is well within the scope of the invention.

Amplification primers for amplifying SSR-type marker loci are a feature of the invention. Another feature of the invention are primers specific for the amplification of SNP domains (SNP markers), and the probes that are used to genotype the SNP sequences.

Typically, molecular markers are detected by any established method available in the art, including, without limitation, allele specific hybridization (ASH) or other methods for detecting single nucleotide polymorphisms (SNP), amplified fragment length polymorphism (AFLP) detection, amplified variable sequence detection, randomly amplified polymorphic DNA (RAPD) detection, restriction fragment length polymorphism (RFLP) detection, self-sustained sequence replication detection, simple sequence repeat (SSR) detection, single-strand conformation polymorphisms (SSCP) detection, isozyme markers detection, or the like. While the exemplary markers provided in the figures and tables herein are SSR or markers, any of the aforementioned marker types can be employed in the context of the invention to identify chromosome segments encompassing genetic element that contribute to superior agronomic performance (e.g., resistance or improved resistance).

In some aspects, the invention provides QTL chromosome intervals, where a QTL (or multiple QTLs) that segregate with FKR resistance are contained in those intervals. A variety of methods well known in the art are available for identifying chromosome intervals. The boundaries of such chromosome intervals are drawn to encompass markers that will be linked to one or more QTL. In other words, the chromosome interval is drawn such that any marker that lies within that interval (including the terminal markers that define the boundaries of the interval) can be used as markers for FKR resistance. Each interval comprises at least one QTL, and furthermore, may indeed comprise more than one QTL. Close proximity of multiple QTL in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifying the same QTL or two different QTL. Regardless, knowledge of how many QTL are in a particular interval is not necessary to make or practice the invention.

In a particular embodiment of the invention, resistance to Fusarium verticillioides was researched and selected for in two recombinant-inbred (RI) populations, as further described in the Examples. The resistant donor NV14FR was crossed with the susceptible parents NV14 and NV35 to create 800 random F2 recombinant inbred lines for each population. A method of selective genotyping (Xu and Vogl, 2000) was employed, where each F2 ear was rated for FKR and the tail regions representing the most susceptible ears and resistant ears would be genotyped with polymorphic SSR markers. 94 plants from the NV14/NV14FR population were selected representing 11.75% of the population or approximately the 5% most resistant and 5% most susceptible ears chosen from a normally distributed bell shaped curve. For the NV35/NV14FR population, 77 plants were selected for maker analysis of the F2 plant tissue. The resulting F3 seeds and donor parents for each population were grown out ear to row in Molokai, Hi. and the resulting F3 mean scores were matched to the F2 genotypes and analyzed to detect significant differences between parent alleles present and the correlating phenotype. In the NV14 population, the correlation of the total mean scores and the parent alleles at the marker phi333597 was significant at the level of p=0.05. The markers umc1350 and dup013 exhibited data supporting a significant QTL in the NV35 population. Four QTLs were identified as being significant at p=0.05 in the NV35 population: umc2013, umc1350, dup013, and umc1665. The significant markers found in these populations were also compared to markers found for the same trait in previous research (Robertson-Hoyt, 2006) (Perez-Brito, 2001) (Jun-Qiang, 2008) to support the presence of a heritable QTL in each genomic region. Three recombinant inbred lines (RILs) from the NV14/NV14FR population were tested in a hybrid combination at 30 locations throughout MN, IL, IA, IN, MI, and WI in 2007. The hybrid performance of all 3 RILs were not significantly different than the isoline hybrid check and all 3 inbreds showed improved resistance for FKR.

The invention is further described with the aid of the following illustrative examples.

### EXAMPLES

### Example 1

### Identification of Parental Line Donor

NV14FR, NV14, NV14HP, and 51×HHP were sent to Sidney, Ill. and Molokai, Hi. in the summer of 2005 for disease screening. The Sidney, Ill. screening was lost due to drought. Visual observations in Molokai, Hi. confirmed the NV14FR had a slight increase in resistance to F. verticillioides infection and was reconfirmed with visual observation in the 2005 winter season in Molokai, Hi. NV14FR was selected as the resistant donor due to the level of resistance it provided in its specific heterotic group and the combining ability in hybrid combinations that it provided.

### Example 2

### Identification of Susceptible Parents

The Mycogen inbred lines NV14 and NV35 were selected as susceptible parents for the populations. The susceptible parents were selected for their consistency to be infected with F. verticillioides, their combining ability in hybrid combinations, and the amount of polymorphic markers remaining between donor parent and susceptible parent. The NV35/NV14FR population was selected based on the greater number of polymorphic markers present, while the NV14/NV14FR population was selected based on the genotypic and phenotypic similarity between parents which will reduce heterosis and hybrid vigor between the RILs but also result in fewer segregating markers remaining This population was designed to study the remaining polymorphic markers that existed from the donor parent in the conversion that resulted in NV14FR, to determine which location the resistance might be coming from.

### Example 3

### Development of Recombinant-Inbred Lines

The NV14/NV14FR F1 seed was made in 2005 in Molokai, Hi. The F1 plants were self pollinated to create F2 seed in the winter of 2005-2006 in Santa Isabel, Puerto Rico. F2 plants were grown in Molokai, Hi. and Fowler, Ind. in the summer of 2006. 400 plants at each location were self pollinated and plant tissue was collected for future marker analysis. The ears in Molokai were naturally infested with F. verticillioides and the F2 ears in Fowler were manually inoculated with F. verticillioides isolates. The ears were ranked on a 1-9 scale for visual presence of infested kernels The rating scale was based on the percent of infected kernels seen on the ear and correlated as: 1=89-99%, 2=78-88%, 3=67-77%, 4=56-66%, 5=45-55%, 6=34-44%, 7=23-33%, 8=12-22%, 9=1-11%. This scale is similar to the 1-7 scale used in previous studies for FKR ratings, where in this study 1 would be the most susceptible or 99% infected kernels and 9 being most resistant or 1% infected kernels. Ninety-four (94) F2 ears were selected for screening with markers. The 94 plants represented 11.75% of the population or approximately the 5% resistant and 5% susceptible tail region on a normally distributed bell shaped curve. The 94 ears were comprised of 43 resistant ears (23 from Molokai and 20 from Fowler) and 51 susceptible ears (29 from Molokai and 22 from Fowler). The F3 plants were grown ear to row in Molokai, Hi. in the winter of 2006 for the final ear rating analysis.

The NV35/NV14FR F1 seed was made in the summer of 2006 in Fowler, Ind. F1 seed was grown in the winter at Molokai, Hi. and F1 plants were self pollinated to make F2 seed. F2 seed was planted at Molokai, Hi. and Fowler, Ind. in the summer of 2007. 400 plants at each location were self pollinated and plant tissue was collected for future marker analysis. The ears in Molokai were naturally infested with F. verticillioides and the F2 ears in Fowler were manually inoculated with F. verticillioides isolates. The ears were ranked on a 1-9 scale for visual presence of infested kernels using the scale listed for the NV14 population. 77(41-Molokai, 36-Fowler) plants were selected for maker analysis of the F2 plant tissue, representing the approximate 5% tail regions of the ear ratings plotted on a bell shaped curve. The F3 plants were grown ear to row in Molokai, Hi. in the winter of 2007 for the final ear rating analysis.

### Example 4

### Polymorphic Markers for Segregating Populations

DNA plant tissue from the NV14/NV14FR and NV35/NV14FR populations, were extracted and quantified from leaf punches of V6 to V8 corn growth stage using DNAEasy 96 Plant Test Kit (Qiagen, Valencia, Calif.). Tissue was collected on 400 F2 field grown plants at both the Molokai and Fowler locations. For DNA quantification, PicoGreen® dye from Molecular Probes, Inc. (Eugene, Oreg.) was diluted 200 fold into 1×TE buffer. In a microtiter plate, 100 μl of the diluted PicoGreen® dye/buffer solution were added into each well followed by 10 μl of each DNA sample or Lambda DNA standards (0, 2.5, 5, and 10 μg/ml). The plate was then agitated on a plate shaker briefly and read using the Spectra Max GEMINIS XK microplate fluorometer from Molecular Devices (Sunnyvale, Calif.). Simple Sequence Repeat (SSR) markers were previously purchased from Applied Biosystems. The sequencing information for the markers is located in the Maize Genetics and Genomics Database (http://www.maizegdb.org/). SSR forward primers were labeled either with 6-FAM, HEX, VIC or NED (blue, green and yellow, respectively) fluorescent tags and synthesized by Applied Biosystems (Foster City, Calif.). PCR was performed in 384-well PCR plates, with each reaction containing 5 ng of genomic DNA, 1.25×PCR buffer (Qiagen, Valencia, Calif.), 0.20 μM of each forward and reverse primer, 1.25 mM MgCl2, 0.015 mM of each dNTP, and 0.3 units of HotStar Taq DNA polymerase (Qiagen, Valencia, Calif.). Amplifications were performed in a GeneAmp PCR System 9700 with 384-dual head module (Applied Biosystems, Foster City, Calif.). Amplification program was as follows: initial activation of Taq at 95° C. for 12 minutes, 40 cycles of 5 sec at 94° C., 15 sec at 55° C., 30 sec at 72° C., and ending with 30 min extension at 72° C. The PCR products for each SSR marker panel were multiplexed together by adding 2 μl of each PCR product to sterile deionized water to make a total volume of 60 μl. 0.8 μl Multiplexed PCR products were stamped into 384-well loading plates containing 5 μl of loading buffer comprised of a 1:100 ratio of GeneScan 500 base pair LIZ size standard and ABI HiDi Formamide (Applied Biosystems, Foster City, Calif.). The samples were then loaded on an ABI Prism 3730xl Automated Sequencer (Applied Biosystems, Foster City, Calif.) for capillary electrophoresis using manufacturer's instructions with a total run time of 36 minutes. Marker data was collected by the ABI Prism 3730xl Automated Sequencer Data Collection software Version 4.0 and extracted by using GeneMapper 4.0 software (Applied Biosystems) for allele characterization and fragment size labeling.

### Example 5

### Disease Screening in Fowler, Ind.

All F2 plants grown at the Fowler, Ind. location were artificially infested with inoculum containing F. verticillioides spore cultures. F. verticillioides inoculum plates were obtained through the following methods. Four symptomatic kernels are excised from air dried corn ears and dipped for 5-10 sec in 70% ETOH before transfer to 1.05% sodium hypochlorite solution for 2 minutes. Kernels blot and air dried for 1 minute before transfer to Petri plates containing filter paper. Approximately 2 ml of sterile water is added for moisture; the plates are wrapped with Parafilm and placed into 25° C./20° C. under fluorescent lighting on a 14/10 hour diurnal cycle incubator for 48 to 72 hours. Germinating hyphe/mycelium/conidia was transferred to media plates for initial isolation. Various media preparations were used to increase the odds of successful culturing of isolates. Media includes: Difco™ Potato Dextrose Agar (Becton, Dickinson and Company) prepared per the manufacturer's instructions and Difco™ PDA amended with 1 ml/L Streptomycin Sulfate BP910-50 (Thermo Fisher Scientific) and finally ½ rate PDA prepared from 19.5 g Difco™ PDA, 7.5 g of Agar BP143-500 (Thermo Fisher Scientific) suspended 1 L dH20 and autoclaved for 15 minutes at 121° C. Cultures are maintained on ½ rate PDA to induce greater sporulation and to lessen mycelial growth. The inoculum plate were grown in a climate controlled room maintained at 23° C. with natural and fluorescent lighting for 14 days prior to storage at 10° C.

After the germination and incubation step, each plate of inoculum was transferred into a solution by pressing them through a wire mesh screen into 500 ml of deionized water and then straining the mixture of water and inoculum through cheese cloth, making sure the inoculum was mixed well and a clean solution was present. A hog vaccinator with a ball pointed needle was connected to an air pressurized jug of inoculum and set to deliver 5 ml of inoculum to each plant. Each plant was inoculated 13-14 days post anthesis which was tracked by marking the flowering date of each plant on its pollination bag. The needle of the hog vaccinator was slowly inserted down the silk channel and pushed into the top of the ear, making sure to rupture kernels at the tip of the ear and not split the ear husk open. Kernels were at the blister stage during this process. After rupturing the kernels with the needle, a 5 ml amount of inoculum was delivered to the ear. The ears were husked back and scored on a scale of 1-9 for F. verticillioides kernel rot symptoms at 45-50 days post flowering.

### Example 6

### Disease Screening in Molokai, Hi.

Inoculation of F. verticillioides in Molokai, Hi. was done by natural infestation. Depending on the need for seeds in future breeding, plants were either open pollinated or hand self pollinated. The plants were left in the field until 40 days post flowering, which was tracked by flowering dates on the pollination bag. At 40 days post flowering, the ear husks were peeled back and the ears were scored on a scale of 1-9 for F. verticillioides kernel rot symptoms.

### Example 7

### Analysis of Phenotypic Data

The F2 ear ratings were summarized and graphed to identify the quantitative or qualitative nature of the FKR trait. The Fusarium resistance trait is thought to be inherited quantitatively, and a normal bell shaped curve would be expected when plotting the ear ratings. From this normally distributed curve, the tail regions (5% most resistant and 5% most susceptible) were identified and selected for marker screening. The F3 ear rating data for both populations was analyzed in JMP 7.0.2 for ANOVA to reject the null hypothesis for both populations and the data is shown in Table 1. JMP 7.0.2 was also used to determine the amount of variation explained by each variable present in the populations. This data is not shown, but will be discussed in the results.

TABLE 1
Variance Components for NV35/NV14FR F3 Ear Ratings

| | Var | % of | | Sqrt(Var |
|---|---|---|---|---|
| Component | Component | Total | Plot% | Comp) |
| RIL | 0.5991397 | 21.0 | 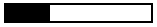 | 0.7740 |
| Rep[RIL] | 0.1717658 | 6.0 | 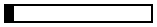 | 0.4144 |
| Observer[RIL, Rep] | 0.1255352 | 4.4 | 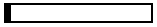 | 0.3543 |
| Within | 1.9558204 | 68.6 | 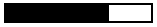 | 1.3985 |
| Total | 2.8522610 | 100.0 | 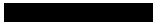 | 1.6889 |

### Example 6

### Analysis of Molecular Marker Data

The 94 F2 plants from the NV14 population were analyzed for the parent alleles present at the 7 informative polymorphic markers which was collected by the ABI Prism 3730xl Automated Sequencer Data Collection software Version 4.0 and extracted by using GeneMapper 4.0 software (Applied Biosystems) for allele characterization and fragment size labeling. The parent alleles were labeled at each marker by labeling the resistant donor allele B,B (NV14FR), the heterozygous allele A,B (NV14/NV14FR) and the susceptible donor allele A,A (NV14). 145 F2 plants from the NV35 population were analyzed for the parent alleles present at the 64 informative markers using the same procedure described for the NV14 population. The parent alleles were labeled at each marker by labeling the resistant donor allele B, B (NV14FR), the heterozygous allele A,B (NV35/NV14FR), and the susceptible parent A,A (NV35) A label of z,z represented a bad gel or unreadable gel for an individual line and marker and these data points were eliminated from the data set. A label of B,D or A,D represented an odd allele not donated by one of the intended parents and these data points were eliminated from the data set. 10 markers of the total 64 markers were ran at a later date than the original set of 54, resulting in several RILs not having enough DNA left in the extraction to amplify and read on the marker analysis, which is represented by a blank in the data set for each RIL and marker.

### Example 7

### Analysis of Phenotype by Genotype Data

The F3 ears were scored for FKR and a total mean score was generated for each Recombinant Inbred Line (RIL) by averaging the scores of all ears and reps. The F3 phenotype total mean score was matched to the F2 marker data for each RIL. The total mean of each RIL and the parent alleles were analyzed for significant differences between total mean scores and significant differences, by using the JMP 7.0.02 procedure Analyze, Fit Y by X, and selecting total mean as the Y response and each marker as the X factor. To compare the means a Tukey-Kramer HSD test was run on each marker. The F3 phenotype data matched to the F2 parent allele data was also run on MAPQTL to map potential QTLs and to also determine the amount of phenotypic variation explained by each marker.

### Example 8

### Analysis of Hybrid Yield Data

Three selections of the NV14/NV14FR population were chosen for yield testing in 2007 at 30 yield trial locations in North America. 25 locations of data were approved for analysis of yield means and yield reports were generated using DowAgroSciences internal database and reporting software. The following traits were included in the report to help evaluate each hybrid: Yield, moisture, percent of plants root lodged, percent of plants stalk lodged, plant height, ear height, dropped ears, top plant integrity, test weight, final population, and flowering date. The focus of the yield mean evaluation will be on the variables of yield, moisture (H20), root lodging, and stalk lodging but all hybrid characteristics will be taken into consideration.

### Example 9

### Verticillioides in the F2 Generation

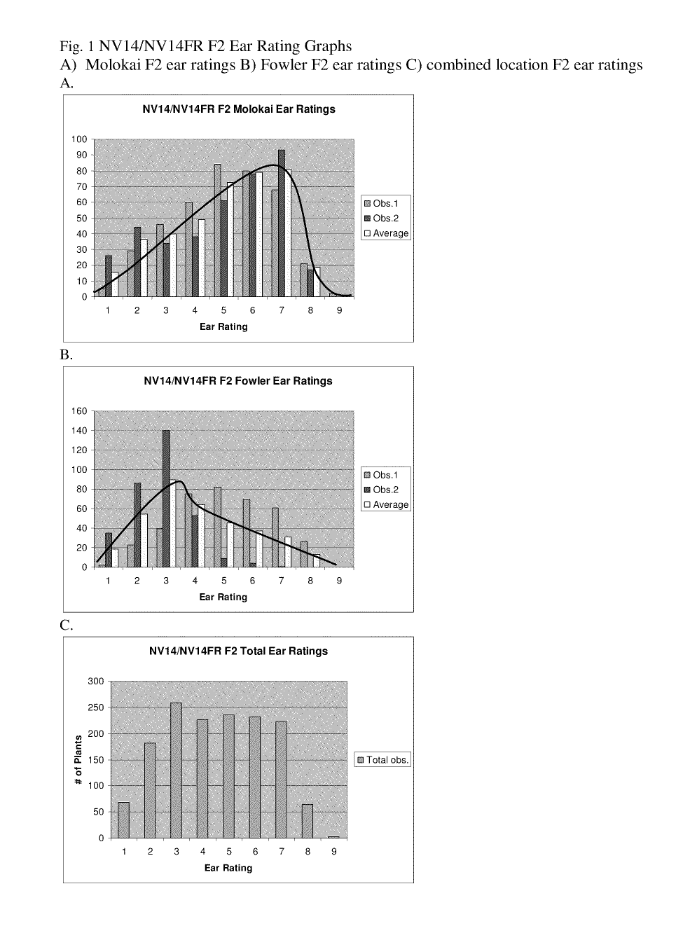

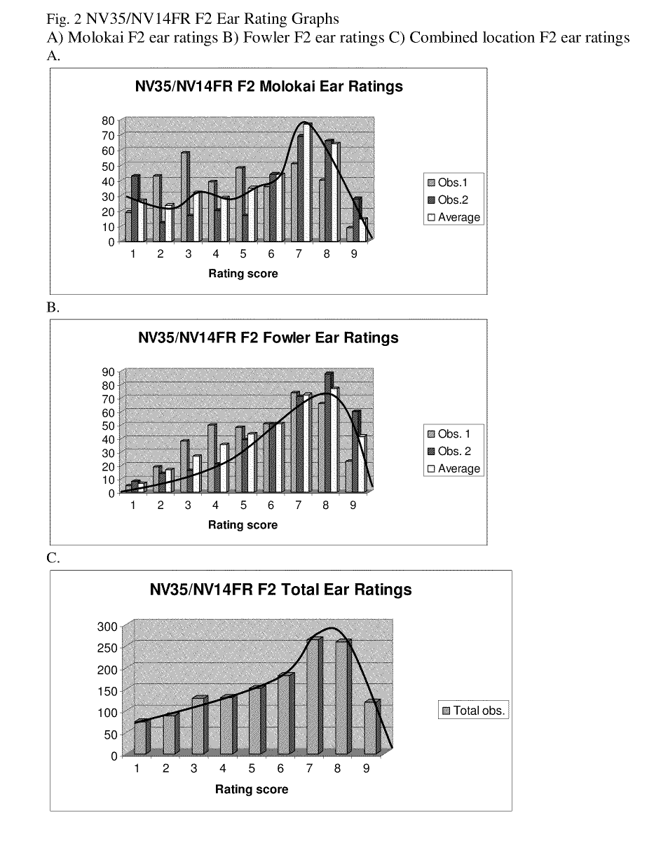

The F2 ears of both populations had good presence of FKR syndromes at the manually inoculated Fowler, Ind. site and the naturally infested Molokai, Hi. site. The natural infestation of the Molokai, Hi. site was favored to help reduce the variability that may be incurred when manually infesting the ears and the ears showed a greater presence of ear rot syndromes in the susceptible ears, possibly due to the environmental factors such as temperature, humidity, and insect vectors. However, when comparing the set of ears using ANOVA and the Tukey-Kramer HSD test, there were no differences seen between the ears selected from Fowler disease screening and the Molokai disease screening. The Fowler and Molokai F2 ears for both populations were scored by two observers and the individual ratings and total ratings were summed and plotted against the rating score to predict the inherent nature of the trait. (see FIGS. 1 and 2). The NV14/NV14FR population had a skewed to the left distribution, while the NV35/NV14FR had a slightly skewed to the right distribution, which could be explained by the increase in heterosis in the NV35/NV14FR population, resulting in more vigorous and healthy plants resulting in higher scores for resistance to FKR. From the NV14/NV14FR population 25 F2 ears were advanced to F3 replicated ear family testing in Molokai, of which 17 had resistant scores on the F2 and eight had susceptible scores on the F2. From the NV35/NV14FR population 145 ears were advanced to F3 replicated ear family testing in Molokai, of which 69 had resistant scores on the F2, 49 had susceptible scores on the F2, and 27 were randomly chosen from mid range scores in the F2 generation.

### Example 10

### Verticillioides in the F3 Generation

The 25 NV14/NV14FR F3 ears were grown out in the winter of 2006 in Molokai, in a randomized complete block design with 4 reps per ear family. The ear families were husked back and phenotype scores were recorded for each individual ear. ANOVA was run to detect the significant differences between each individual RIL and the reps. There was a significant difference between repetitions 1 and 2, and repetitions 3 and 4 found. This difference was due to the fact that reps 1 and 2 were cut back and hand pollinated to increase seed for future tests while reps 3 and 4 were open pollinated. (Table 3) When plants were hand pollinated the ear would have been covered with a pollen bag after pollination and this would reduce the amount of F. verticillioides horizontally transmitted to the silks via air borne conidia. There was only one significant difference seen between the mean scores of the RILs which was between F3 ear families −147 and −287. (Table 4) Only 1 observer scored the ears in the NV14/NV14FR population so only RIL and rep could be used to estimate variance components in this population.

Table 3
NV14/NV14FR Rep Analysis
Oneway Analysis of F3 Ear rating By Rep #

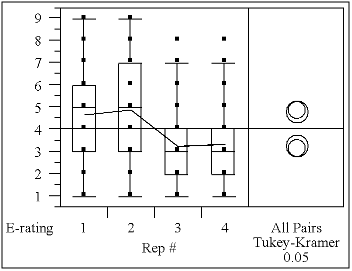

Means Comparisons
Comparisons for all pairs using Tukey-Kramer HSD (p = .05)

| Rep # | | | Mean |
|---|---|---|---|
| 2 | A | | 4.8666667 |
| 1 | A | | 4.7677419 |
| 4 | | B | 3.2564103 |
| 3 | | B | 3.1090909 |

Levels not connected by same letter are significantly different.

TABLE 4
Significant Difference of NV14/NV14FR F3 Ear Rating Means
Comparisons for all pairs using Tukey-Kramer HSD

| RIL Number | | | | Mean |
|---|---|---|---|---|
| E0006728-6914B (Res. Check) | A | | | 6.89 |
| ZW06EW011938.1572 | A | B | C | 4.80 |
| ZQ06EQ463937.287 | | B | | 4.65 |
| ZW06EW011938.1520 | | B | C | 4.47 |
| ZQ06EQ463910.186 | | B | C | 4.45 |
| (NV14FR) | | B | C | 4.45 |
| ZW06EW011938.1608 | A | B | C | 4.43 |
| ZQ06EQ463870.021 | | B | C | 4.40 |
| ZW06EW011938.1708 | | B | C | 4.33 |
| ZQ06EQ463878.065 | | B | C | 4.29 |
| ZQ06EQ463943.335 | | B | C | 4.21 |
| ZQ06EQ463910.196 | | B | C | 4.19 |
| ZW06EW011938.1539 | | B | C | 4.05 |
| ZW06EW011938.1646 | | B | C | 4.04 |
| ZW06EW011938.1681 | | B | C | 4.00 |
| ZQ06EQ463910.194 | | B | C | 3.92 |
| ZW06EW011938.1687 | | B | C | 3.80 |
| ZQ06EQ463907.177 | | B | C | 3.68 |
| ZW06EW011938.1746 | | B | C | 3.63 |
| ZW06EW011938.1527 | | B | C | 3.56 |
| ZW06EW011938.1553 | | B | C | 3.44 |
| ZW06EW011938.1507 | | B | C | 3.43 |
| (NV14) | | B | C | 3.23 |
| ZW06EW011938.1454 | | B | C | 3.13 |
| ZW06EW011316 | | B | C | 3.11 |
| ZW06EW011938.1529 | | B | C | 3.00 |
| ZW06EW011938.1489 | | B | C | 2.88 |
| ZW06EW011938.1416 | | B | C | 2.67 |
| ZQ06EQ463903.147 | | | C | 2.63 |

Levels not connected by same letter are significantly different.

The 145 NV35/NV14FR F3 ears were grown in the winter of 2007 in Molokai, in a randomized complete block design with 2 reps per ear family. Only four plants were hand pollinated in the first rep leaving all remaining plants open pollinated, which resulted in no significance seen between repetitions in this population. The ears were harvested by row and each ear was scored by an observer in Molokai and in Fowler. ANOVA was run on the ear rating data and no significant differences were seen between the reps but there was significant difference seen between observers. However, the amount of variation explained by the observers when nested with the RIL was only 3.4% of the variation while the amount explained between each RIL was 23.3% with the remaining 73.3% variation occurring within each row of the F3 ear families, so both observers ratings were averaged together as the total mean for each RIL.

### Example 11

### Significant Markers and MapQTL

The F3 ear rating total means and F2 genotype data were matched together for each RIL of each population. For each informative polymorphic marker, the parent alleles and their corresponding mean scores were analyzed for the total variation and the amount of variation explained by the reps, observers, and locations, as shown in Table 1 (above) and Table 2.

TABLE 2
Variance Components for NV14/NV14FR F3 Ear Ratings

| | Var | | | Sqrt(Var |
|---|---|---|---|---|
| Component | Component | % of Total | Plot % | Comp) |
| RIL | 0.3365364 | 8.7 | 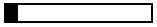 | 0.5801 |
| Rep#[RIL] | 1.2063296 | 31.1 | 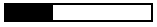 | 1.0983 |
| Within | 2.3401013 | 60.3 | 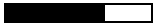 | 1.5297 |
| Total | 3.8829674 | 100.0 | 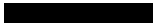 | 1.9705 |

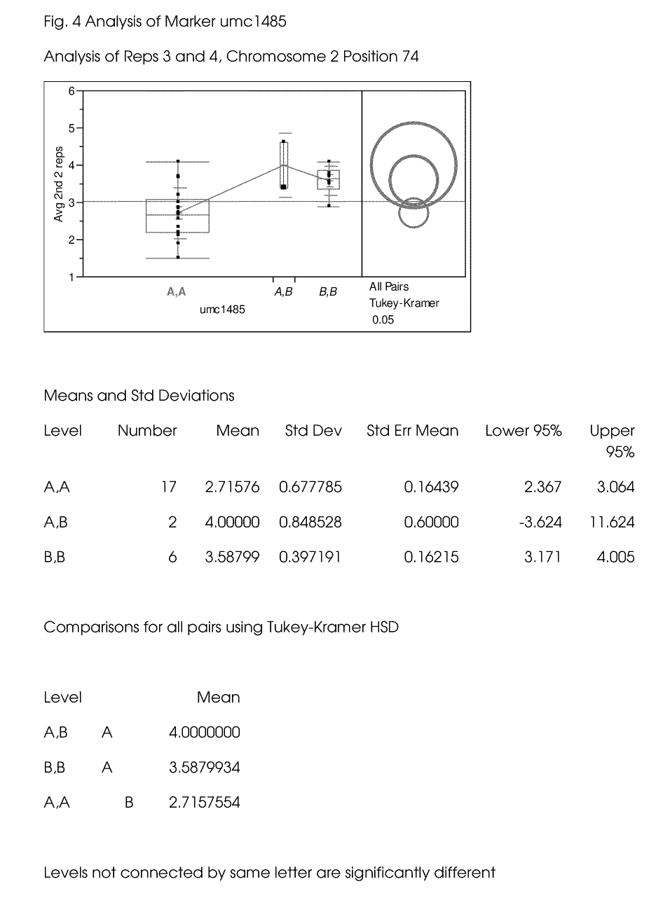

The mean scores for each parent allele of a RIL were analyzed using the Fit Y by X procedure in JMP 7.0.2, and significant differences between parent alleles were identified using the Tukey-Kramer HSD test. In the NV14/NV14FR population, two markers were identified as having significant differences between parent alleles and phenotype correlation indicating the possible presence of a QTL at these areas of the maize genome. On chromosome 5, position 88 cM, the marker phi333597 was significant at p=0.05 (FIG. 2.3). At this marker, the parent allele correlating to resistance came from the NV14FR resistant donor (B,B). With the very limited marker coverage in this population, it was not possible to link phi33597 to another marker in MapQTL, but when comparing these significant markers to the results of Robertson-Hoyt (2006), this marker is located in a region similar to the marker umc2111 (Table 5). In the research of Robertson-Hoyt, the marker umc2111 was determined to explain 3.8% of the phenotypic variation in that study. There was a significant difference between reps in the field for the NV14 population, where reps 1 and 2 could be grouped together, but they were significantly different than reps 3 and 4, which could be grouped together. An ANOVA and Tukey-Kramer HSD test was run on the average means of reps 3 and 4 grouped together. The marker 1485, on chromosome 2, position 74 cM showed up as having significant differences between the mean scores for the parent alleles when grouping these reps together (see FIG. 4). The parent allele confirming resistance was from the NV14FR (B,B) resistant donor parent and it could not be linked to any other markers using MapQTL, however the mean scores for ear rot would indicate that the gene action is dominant at this locus as both B,B and A,B genotypes correlated to better resistance scores.

TABLE 5
List of Markers Associated with FKR Resistance

| | | | | | Year |
|---|---|---|---|---|---|
| genprobename | locusname | bin | IBM neigh. | FKR Author | Reported |
| p-umc1485 | umc1485 | 2.04 | 329.6 | VanOpdorp | 2009 |
| p-umc1355 | umc1355 | 5.03 | 281.2 | Robertson-Hoyt | 2006 |
| p-umc2111 | umc2111 | 5.05 | | Robertson-Hoyt | 2006 |
| p-phi333597 | phi333597 | 5.05 | 394.4 | VanOpdorp | 2009 |
| p-umc1524 | umc1524 | 5.06 | 493.5 | Robertson-Hoyt | 2006 |
| p-umc2013 | umc2013 | 5.07 | 571.66 | VanOpdorp | 2009 |
| p-umc1388 | umc1388 | 6.05 | 302 | Perez-Brito | 2001 |
| p-nc012 | pdk1 | 6.05 | 323.5 | Perez-Brito | 2001 |
| p-phi078 | pdk1 | 6.05 | 323.5 | Perez-Brito | 2001 |
| p-umc1388 | umc1388 | 6.05 | 302 | Perez-Brito | 2001 |
| p-umc2375 | umc2375 | 6.06 | 431.04 | Robertson-Hoyt | 2006 |
| p-umc2375 | umc2375 | 6.06 | 431.04 | Robertson-Hoyt | 2006 |
| p-umc132 | umc132a(chk) | 6.07 | 444.2 | Perez-Brito | 2001 |
| p-umc1350 | umc1350 | 6.07 | 504.8 | VanOpdorp | 2009 |
| p-umc1350 | umc1350 | 6.07 | 504.8 | VanOpdorp | 2009 |
| p-bnlg1740 | bnlg1740 | 6.07 | 510.6 | Robertson-Hoyt | 2006 |
| p-umc1412 | umc1412 | 7.04 | 518.9 | VanOpdorp | 2009 |
| | dup013 | 7.04 | | VanOpdorp | 2009 |
| p-umc1460 | umc1460 | 8.04 | 304.2 | Jun-Qiang | 2008 |
| p-umc1562 | umc1562 | 8.05 | 353.3 | Jun-Qiang | 2008 |
| p-umc1665 | umc1665 | 8.05 | 390.26 | VanOpdorp | 2009 |

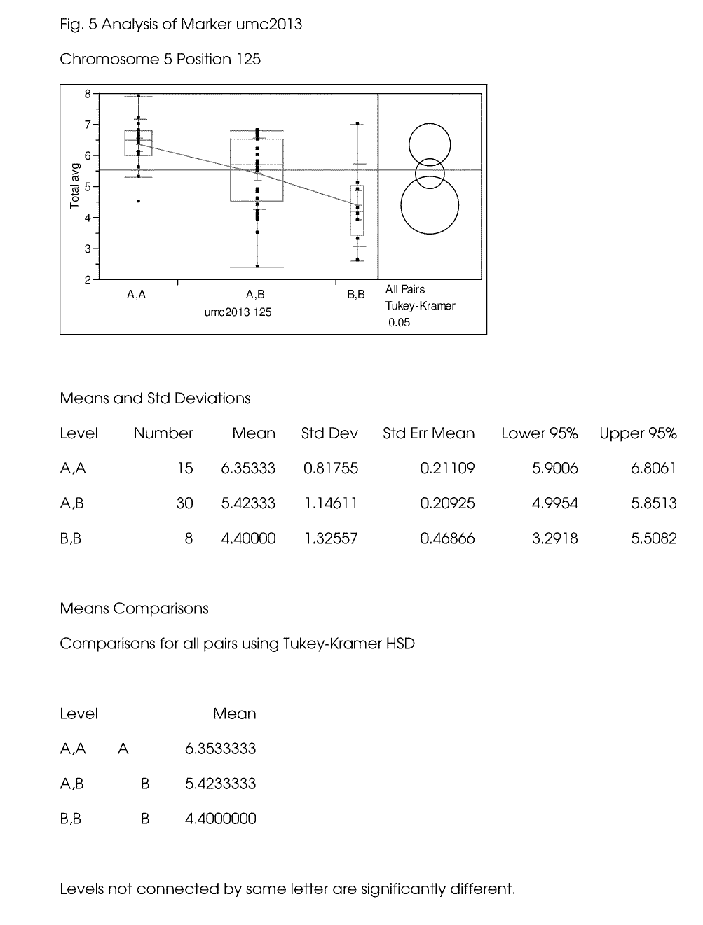

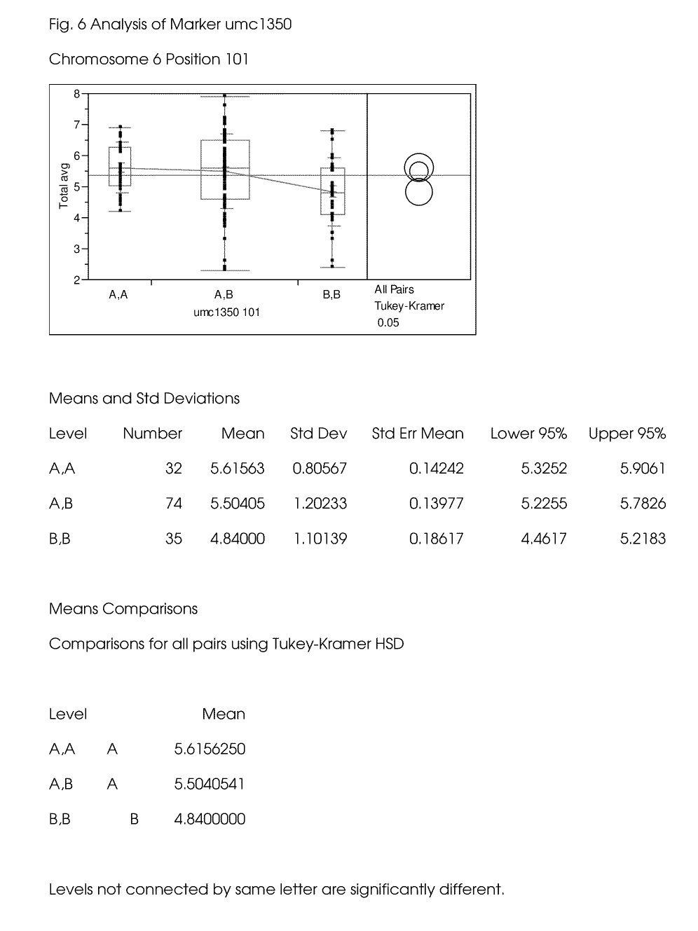

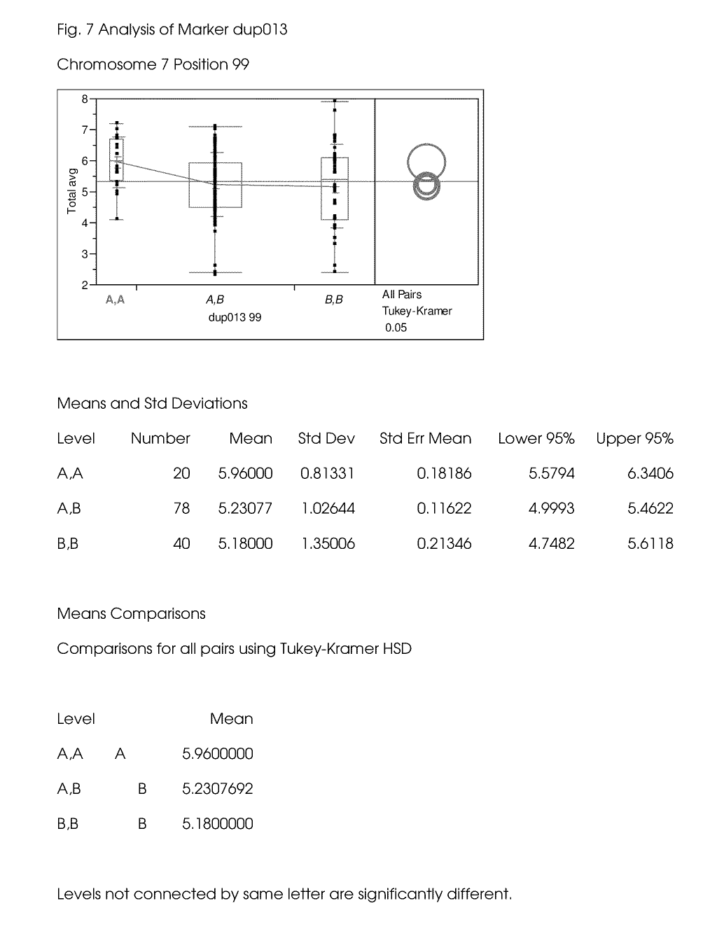

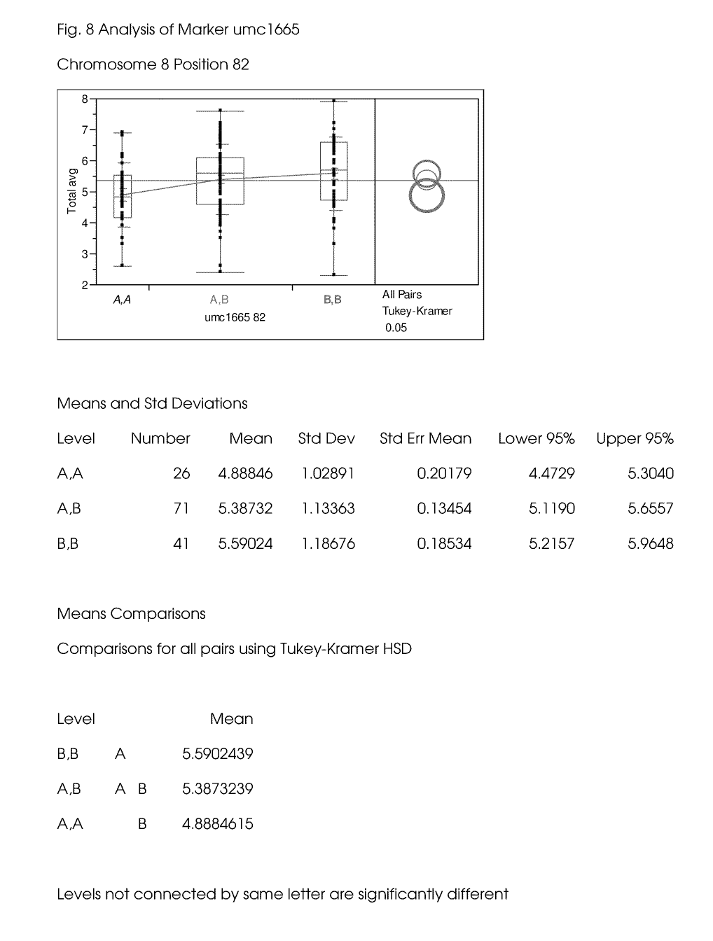

In the NV35/NV14FR population, four markers were found to have significant differences between the total mean scores of the different parent alleles. The Fit Y by X procedure was used in JMP 7.0.2 to generate the summary of means for each genotype at each marker and a Tukey-Kramer HSD test was run on the mean data to determine which markers had significant differences between the mean scores for each genotype. The data supports markers umc 1350 and dup013 as significant markers. Umc2013 and umc1665 also had significant differences between the genotype and mean scores for ear rot at p=0.05 (FIGS. 5-8). This data would predict that the markers umc2013, umc1350, dup013, and umc1665 are located in regions which are associated with resistance to FKR. The marker umc2013 was also found to be significant at p=0.01. All markers were run in MapQTL to match up linkage groups for the significant markers at a 3.6 LOD threshold of a 1000 permutation test. Although no markers met the criteria in MapQTL, umc1350 and dup013 had LOD values to indicate that these markers could possibly explain some of the phenotypic variation associated with FKR (Table 6) The markers umc2013 and umc1665 were unable to be mapped due to the lack of a nearby polymorphic marker in the population. The markers umc2013, umc1350, and umc1665 located in chromosomal bins 5.07, 6.07, and 8.05, respectively, were also located in regions near markers previously identified by Robertson-Hoyt (2006) and Perez-Brito (2001). The significant markers from this study and the markers in close proximity to these found in other studies is listed in Table 2.5

TABLE 6
MapQTL analysis of significant markers

| | | | | | | | | | | |
|---|---|---|---|---|---|---|---|---|---|---|
| map | lod | iter | mu_A | mu_H | mu_B | var | % expl | add | dom | locus |

linkage group 7 (Chr._6_(LOD = 3)):

| | | | | | | | | | | |
|---|---|---|---|---|---|---|---|---|---|---|
| 0 | 1.44 | 4 | 5.55 | 5.50 | 4.96 | 1.22 | 4.5 | 0.291 | 0.241 | umc1490 |
| 5 | 2 | 6 | 5.60 | 5.52 | 4.87 | 1.19 | 6.8 | 0.366 | 0.281 | |
| 10 | 2.51 | 5 | 5.62 | 5.53 | 4.83 | 1.18 | 7.9 | 0.397 | 0.303 | |
| 10.4 | 2.54 | 5 | 5.62 | 5.53 | 4.83 | 1.18 | 7.9 | 0.398 | 0.303 | umc1350 |

linkage group 9 (Chr._7B_(LOD = 3)):

| | | | | | | | | | | |
|---|---|---|---|---|---|---|---|---|---|---|
| 0 | 1.89 | 5 | 6.03 | 5.27 | 5.21 | 1.20 | 6.2 | 0.411 | −0.347 | dup013 |
| 5 | 1.73 | 8 | 6.02 | 5.26 | 5.21 | 1.20 | 6.7 | 0.406 | −0.361 | |
| 10 | 1.55 | 12 | 6.00 | 5.24 | 5.22 | 1.19 | 6.8 | 0.387 | −0.370 | |
| 15 | 1.36 | 16 | 5.96 | 5.22 | 5.25 | 1.20 | 6.4 | 0.354 | −0.382 | |
| 20 | 1.18 | 20 | 5.91 | 5.20 | 5.29 | 1.21 | 5.9 | 0.308 | −0.396 | |
| 25 | 1.02 | 19 | 5.85 | 5.20 | 5.33 | 1.22 | 5.1 | 0.261 | −0.390 | |
| 30 | 0.87 | 14 | 5.78 | 5.21 | 5.34 | 1.23 | 4 | 0.222 | −0.353 | |
| 35 | 0.74 | 9 | 5.72 | 5.23 | 5.34 | 1.24 | 3 | 0.189 | −0.301 | |
| 40 | 0.64 | 5 | 5.66 | 5.25 | 5.34 | 1.25 | 2.2 | 0.160 | −0.249 | |
| 40.4 | 0.63 | 5 | 5.66 | 5.25 | 5.34 | 1.25 | 2.1 | 0.158 | −0.245 | umc1671 |

3.6 LOD threshold in MAPQTL, 1000 permutation test

### Example 12

### Yield Data Summary

Resistant RILs were selected from the NV14/NV14FR population to be crossed to an elite tester line, and the hybrids were yield tested in 2007 at 30 locations in North America including the iso line check. There was no significant difference seen between all three hybrids and the iso line check across 25 locations of data at both LSD (0.05) and LSD (0.10), as shown by the yield data in Table 7. The −65, −287, and −1539 selection all had total mean scores for FKR resistance higher than the NV14 and NV14HP check (see Table 4), but only the −287 at p=0.20 was significantly different than the iso line checks. With the indication of improved F. verticillioides resistance in the inbred line and competitive or increased hybrid performance in the hybrid, the −65, −287, and 1539 selections can all be utilized for an improved FKR resistant NV14 conversion to be used in breeding and or commercial sale of hybrids. The −65 selection is a highly suitable inbred based on parent alleles present at the significant marker regions and performance in yield testing. The −287 is another suitable candidate for the converted FKR resistant inbred, based on the favorable parent alleles at significant marker regions and total mean score for FKR. The yield data on −287 is competitive based on the percent root lodging (P_RL) and percent stalk lodging (P_SL).

TABLE 7
NV14/NV14FR 2007 Yield Means

| Ent | Name | Yield | Yield #Plots | H2O | P_SL |
|---|---|---|---|---|---|
| 40 | TESTER1HP//NV14/NV14FR-1539 | 208.62 | 25 | 18.21 | 0.18 |
| 39 | TESTER1HP//NV14/NV14FR-287 | 206.97 | 25 | 17.63 | 1.09 |
| 38 | TESTER1HP///NV14/NV14FR-65 | 212.26 | 25 | 17.68 | 0.48 |
| 37 | TESTER1HP/NV14 | 207.36 | 25 | 17.01 | 0.06 |

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What may be claimed is:

1. A method of genetic marker assisted selection of Fusarium Ear Rot (FKR) resistance in maize plants, comprising:
a.) isolating DNA from the maize plants;
b.) assessing the DNA to identify plants having one or more of the SSR genetic markers selected from the group consisting of phi333597, umc2013, umc1350, dup013, umc1665, and umc1412; and
c.) selecting the plants having the Fusarium Ear Rot resistance.

2. The method of claim 1, wherein the DNA is assessed to identify plants having a marker within 1 centimorgan of one or more of the SSR genetic markers selected from the group consisting of phi333597, umc2013, umc1350, dup013, umc1665, and umc1412.

3. A method of identifying a first maize plant or germplasm that displays resistance or improved resistance to FKR, the method comprising detecting in the first maize plant or germplasm at least one allele of a one or more genetic markers associated with the FKR resistance one or more of the genetic markers selected from the group consisting of phi333597, umc2013, umc1350, dup013, umc1665, and umc1412.

4. The method of claim 3, wherein the germplasm is a maize line or maize variety.

5. The method of claim 3, wherein the detecting comprises detecting at least one allelic form of a polymorphic simple sequence repeat (SSR).

6. The method of claim 3, wherein the detecting comprises amplifying one or more of the genetic markers or a portion of the one or more genetic markers, and detecting the resulting amplified marker amplicon.

7. The method of claim 6, wherein the amplifying comprises employing a polymerase chain reaction (PCR) or ligase chain reaction (LCR) with a nucleic acid isolated from the first maize plant or germplasm as a template in the PCR or LCR.

8. The method of claim 3, wherein the at least one allele comprises two or more alleles.

9. The method of claim 3, wherein the one or more genetic markers are determined using the mapping population from the cross between NV14FR and NV14.

10. The method of claim 3, wherein the one or more genetic markers are determined using the mapping population from the cross between NV14FR and NV35.

11. The method of claim 3, further comprising selecting the first maize plant or germplasm, or selecting a progeny of the first maize plant or germplasm comprising the at least one allele of a genetic marker that is associated with the resistance or improved resistance to FKR.

12. The method of claim 11, further comprising crossing the selected first maize plant or germplasm with a second maize plant or germplasm.
